(19) United States
(12) Patent Application Publication
(10) Pub. No.: US 2014/0135639 A1
(43) Pub. Date: May 15, 2014

(54) Adaptive Acoustic Signal Filtering for Respiration Monitoring System

(71) Applicants: Yungkai Kyle LAI, Irvine, CA (US); Yongji FU, Cary, NC (US)

(72) Inventors: Yungkai Kyle LAI, Irvine, CA (US); Yongji FU, Cary, NC (US)

(21) Appl. No.: 13/675,014

(22) Filed: Nov. 13, 2012

(51) Int. Cl. A61B 5/08

(52) U.S. Cl. CPC A61B 5/0803
USPC 600/529

(57) ABSTRACT

An adaptive acoustic signal filter for a respiration monitoring system includes a filter stage and a cutoff frequency adapter. The filter stage applies a cutoff frequency to an input acoustic signal waveform containing respiration and heart sound components in a filtering operation to produce a filtered acoustic signal waveform from which heart sound components have been removed. The adapter then performs cutoff frequency optimization tests on the filtered signal waveform and determines from the tests whether adjustment of the cutoff frequency is indicated. These tests assess whether the filtering operation struck a proper balance between removing heart sound components and preserving respiration sound components in the filtered signal waveform. If adjustment of the cutoff frequency is indicated, the adapter adjusts the cutoff frequency and the adjusted cutoff frequency is provided to the filter stage for application in a next filtering operation performed on the input signal waveform.

### BACKGROUND OF THE INVENTION

The present invention relates to respiration monitoring and, more particularly, to filtering of an acoustic signal to isolate respiration sounds.

In acoustic respiration monitoring, estimates of respiration parameters, such as respiration rate and inspiration to expiration ratio (I:E), are computed by analyzing an acoustic signal captured by a microphone placed on a human body. Before respiration parameters can be computed, however, respiration sounds in the acoustic signal must be disambiguated from heart sounds and other noise.

Because heart sounds usually have frequencies below 100 Hz whereas respiration sounds are usually concentrated above 200 Hz, one conventional approach to removing heart sounds and other noise from the acoustic signal applies a highpass or bandpass filter having a cutoff frequency between these frequencies to the raw signal. However, sound harmonics or muscle movements may cause some heart sound and other noise components to seep through the filter and continue to obscure respiration sound components of the acoustic signal.

Moreover, because heart rate is normally in the range of 60-100 beats per minute whereas respiration rate is normally in the range of 14-20 breaths per minute, another approach to removing heart sound is to apply a lowpass filter having a fixed cutoff frequency between these two rates to an acoustic signal waveform (usually an energy envelope) computed from the raw signal. However, different individuals have different heart and respiration rates, and respiration and heartbeats are rarely periodic when observed in the same individual over a sustained period and oftentimes do not have a single dominant frequency. This is especially true in mobile monitoring applications. As a result, for optimal acoustic respiration monitoring, the lowpass filter must have a cutoff frequency judiciously set to remove heart sound components from the waveform to the extent possible without removing respiration sound components. Yet since the frequency composition of respiration sound components is not determinable with a high degree of confidence until after the heart sound components are removed, setting an optimal cutoff frequency presents a major technical challenge.

One way to estimate an optimal cutoff frequency is to perform a Fourier analysis on the acoustic signal waveform that identifies a fundamental frequency representing the heartbeat. However, variation of the heartbeat, respiration and other signal components over time may result in the appearance of stray peaks in the Fourier transform that obscure the dominant peak at the fundamental frequency. This may make it difficult to estimate the fundamental frequency representing the heartbeat with a high degree of confidence.

Another way to estimate an optimal cutoff frequency is to perform autocorrelation on the acoustic signal waveform to identify a fundamental periodicity representing the heartbeat in a series of peaks and troughs. However, the variation of the heartbeat, respiration and other signal components over time may prevent autocorrelation from yielding a single maximal peak. Moreover, when peaks representing the heartbeat are intermingled with cross-correlation peaks representing respiration and other sounds, it may be difficult to estimate the heartbeat period with a high degree of accuracy.

Faced with these problems in estimating an optimal cutoff frequency for the lowpass filter, the cutoff frequency may simply be set well below what is the best guess for the optimal cutoff frequency to ensure that most heart sound components are removed from the acoustic signal waveform. However, adding such a margin of error may inadvertently result in removal of respiration sound components.

### SUMMARY OF THE INVENTION

The present invention provides adaptive acoustic signal filtering for a respiration monitoring system. An adaptive acoustic signal filter for such a system has a filter stage operatively coupled with a cutoff frequency adapter. The filter stage applies a cutoff frequency to an input acoustic signal waveform containing respiration and heart sound components in a filtering operation to produce a filtered acoustic signal waveform from which heart sound components have been removed. The frequency adapter then performs one or more cutoff frequency optimization tests on the filtered signal waveform and determines from the tests whether adjustment of the cutoff frequency is indicated. These tests assess whether the filtering operation struck a proper balance between removing heart sound components and preserving respiration sound components in the filtered signal waveform. If adjustment of the cutoff frequency is indicated, the frequency adapter adjusts the cutoff frequency and the adjusted cutoff frequency is provided to the filter stage for application in a next filtering operation performed on the input signal waveform. On the other hand, if adjustment of the cutoff frequency is not indicated, the cutoff frequency is considered optimal and continues to be used in filtering the input signal waveform.

In one aspect of the invention, an adaptive acoustic signal filter for a respiration monitoring system comprises a filter stage configured to apply a cutoff frequency to an input acoustic signal waveform containing respiration and heart sound components in a filtering operation to produce a filtered acoustic signal waveform from which heart sound components have been removed; and a cutoff frequency adapter operatively coupled with the filter stage and configured to perform one or more cutoff frequency optimization tests on the filtered signal waveform, determine from the tests whether adjustment of the cutoff frequency is indicated and selectively adjust the cutoff frequency and provide the adjusted cutoff frequency to the filter stage for application in a next filtering operation performed on the input signal waveform depending on whether adjustment of the cutoff frequency is indicated.

In some embodiments, the cutoff frequency optimization tests assess whether the filtering operation struck a proper balance between removing heart sound components and preserving respiration sound components in the filtered signal waveform.

In some embodiments, the cutoff frequency optimization tests comprise a test of residual heart sound presence in the filtered signal waveform.

In some embodiments, the residual heart sound presence test determines whether actual times between peaks in the filtered signal waveform fall within a range of expected times between heartbeats.

In some embodiments, the cutoff frequency optimization tests comprise a test of slopes of peaks in the filtered signal waveform.

In some embodiments, the cutoff frequency optimization tests comprise a test of smoothness of the filtered signal waveform.

In some embodiments, the cutoff frequency is adjusted using a monotonously decreasing function.

In some embodiments, the filter is a lowpass filter.

In some embodiments, the input signal waveform comprises an energy envelope computed from a raw acoustic signal.

In some embodiments, the filter stage and the cutoff frequency adapter comprise software instructions executed by a processor.

In another aspect of the invention, an adaptive acoustic signal filtering method for a respiration monitoring system comprises the steps of applying by the system to an input acoustic signal waveform containing respiration and heart sound components in a filtering operation a cutoff frequency to produce a filtered acoustic signal waveform from which heart sound components have been removed; performing by the system one or more cutoff frequency optimization tests on the filtered signal waveform; determining by the system from the tests whether adjustment of the cutoff frequency is indicated; and selectively adjusting the cutoff frequency and providing by the system the adjusted cutoff frequency to the filter for application in a next filtering operation performed on the input signal waveform depending on whether adjustment of the cutoff frequency is indicated.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a respiration monitoring system in some embodiments of the invention.

FIG. 2 shows an adaptive acoustic signal filter in some embodiments of the invention.

FIG. 3 shows an adaptive acoustic signal filtering method in some embodiments of the invention.

FIG. 4 shows a residual heart sound presence test in some embodiments of the invention.

### DETAILED DESCRIPTION OF A PREFERRED EMBODIMENT

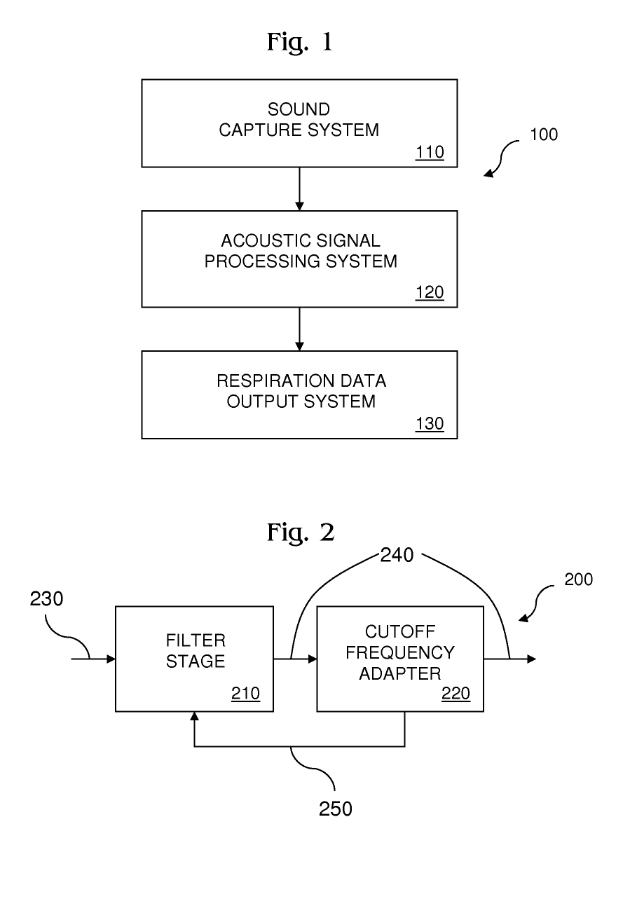

FIG. 1 shows a respiration monitoring system 100 in some embodiments of the invention. Monitoring system 100 includes a sound capture system 110, an acoustic signal processing system 120 and a respiration data output system 130, which are communicatively coupled in series.

Capture system 110 continually detects body sounds, such as respiration and heart sounds, at a detection point, such as the trachea, chest or back of a person being monitored, and continually transmits a raw acoustic signal containing the detected body sounds to processing system 120. Capture system 110 may include, for example, a microphone positioned on the body of a human subject that detects body sounds, as well as amplifiers, filters an analog/digital converter and/or automatic gain control that generate a raw acoustic signal embodying the detected body sounds.

Processing system 120, under control of a processor executing software instructions, receives the raw acoustic signal from capture system 110 and generates estimates of one or more respiration parameters for the subject being monitored for different time segments of the raw acoustic signal. In some embodiments, monitored respiration parameters include respiration rate, fractional inspiration time and/or inspiration to expiration time ratio (I:E).

When processing system 120 receives the acoustic signal from capture system 110, processing system 120 first computes an acoustic signal waveform that is an energy envelope of the raw acoustic signal, to improve signal quality. The energy envelope may be computed, for example, as the biased or unbiased standard deviation of the raw acoustic signal over a small number of data samples. The loudness of sounds is generally proportional to the amplitudes of data points in the energy envelope. Thus, troughs in the energy envelope represent quiet times and peaks or spikes in the energy envelope represent loud times.

Moreover, when processing system 120 first receives the raw acoustic signal from capture system 110, the signal is “mixed”, meaning that respiration sounds are intermingled with heart sounds so as to be unrecoverable. After energy envelope computation, processing system 120 filters the acoustic signal waveform to remove heart sounds, which “clears” the acoustic signal waveform and renders the respiration sounds recoverable. Processing system 120 can then proceed to recover the respiration sounds and generate estimates of one or more respiration parameters.

In some embodiments, processing system 120 applies additional filter prior to energy envelope computation to suppress heart sounds and noise in the raw acoustic signal that is outside the frequency range of interest.

In some embodiments, processing system 120 performs at least some of the processing operations described herein in custom logic rather than software.

Output system 130 has a display screen for displaying respiration information determined using respiration parameter estimates received from processing system 120. In some embodiments, output system 130, in addition to a display screen, has an interface to an internal or external data management system that stores respiration information determined using respiration parameter estimates received from processing system 120 and/or an interface that transmits such information to a remote monitoring device, such as a monitoring device at a clinician facility. Respiration information outputted by output system 130 may include respiration parameter estimates received from processing system 120 and/or information derived from respiration parameter estimates, such as a numerical score or color-coded indicator of present respiratory health status.

In some embodiments, capture system 110, processing system 120 and output system 130 are part of a portable ambulatory monitoring device that monitors a person's respiratory well being in real-time as the person goes about daily activities. In other embodiments, capture system 110, processing system 120 and output system 130 may be part of separate devices that are remotely coupled via wired or wireless communication links.

FIG. 2 shows an adaptive acoustic signal filter 200 in some embodiments of the invention. Filter 200 is a component of processing system 120. Filter 200 includes a filter stage 210 operatively coupled with a cutoff frequency adapter 220. Filter 200 receives as an input acoustic signal waveform 230 the energy envelope computed by an earlier component of processing system 120 from the raw acoustic signal. Input signal waveform 230 contains both respiration sounds and heart sounds. Filter 200 performs an adaptive acoustic signal filtering method to remove heart sound components from input signal waveform 230, converting input signal waveform 230 into a filtered acoustic signal waveform 240 containing a filtered energy envelope from which respiration sound components are recoverable by later components of processing system 120.

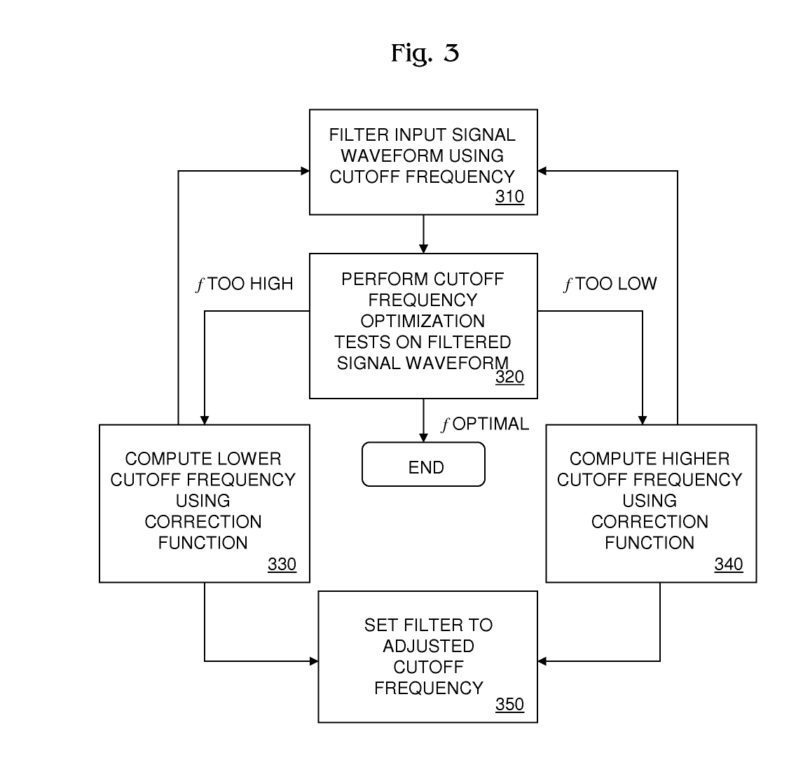

FIG. 3 shows an adaptive acoustic signal filtering method performed by filter 200 in some embodiments of the invention. Filter stage 210 continually receives input signal waveform 230. Filter stage 210 applies to input signal waveform 230, in a lowpass filtering operation, a cutoff frequency to produce filtered signal waveform 240 from which at least some heart sound components have been removed (310) and provides filtered signal waveform 240 to frequency adapter 220. Frequency adapter 220 performs cutoff frequency optimization tests on filtered signal waveform 240 (320) to determine whether adjustment of the cutoff frequency is indicated. These tests assess whether the filtering operation performed on input signal waveform 230 struck a proper balance between removing heart sound components and preserving respiration sound components. If the tests suggest that too many residual heart sound components remain in filtered signal waveform 240, the inference is drawn that the cutoff frequency is too high. In that event, frequency adapter 220 computes a lower cutoff frequency by applying a correction function (330) and sends an instruction 250 to filter stage 210 causing filter stage 210 to set the cutoff frequency to the lower cutoff frequency for the next filtering operation performed on input signal waveform 230. If the tests suggest that too many respiration sound components are missing from filtered signal 240, the inference is drawn that the cutoff frequency is too low. In that event, frequency adapter 220 computes a higher cutoff frequency by applying a correction function (340) and sends an instruction 250 to filter stage 210 causing filter stage 210 to set the cutoff frequency to the higher cutoff frequency for the next filtering operation performed on input signal waveform 230. If the tests suggest that the filtering operation performed on input signal waveform 230 struck a proper balance between removing heart sounds and preserving respiration sounds, the cutoff frequency is considered optimal and continues to be used in filtering of input signal waveform 230.

The cutoff frequency optimization tests performed on filtered signal waveform 240 to determine whether adjustment of the cutoff frequency is indicated may include, without limitation, the one or more of the following:

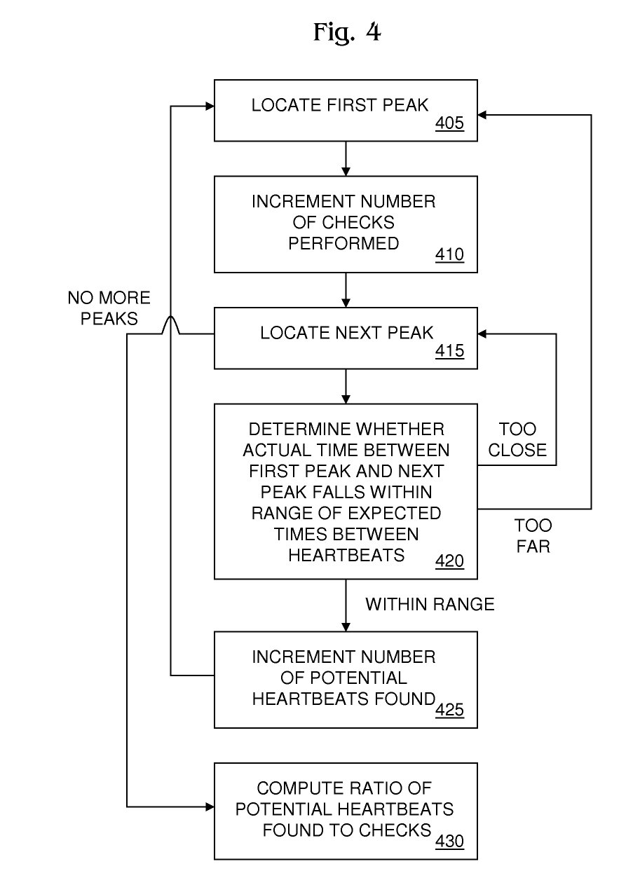

Residual Heart Sound Presence Test. The residual heart sound presence test determines whether and the extent to which actual times between peaks in filtered signal waveform 240 fall within a range of expected times between heartbeats. If the actual time between peaks in filtered signal waveform 240 are within a range of expected times, the peaks potentially represent heartbeats that were not removed in the filtering operation. If there are too many of these residual heartbeats in filtered signal waveform 240, the cutoff frequency may be considered too high. FIG. 4 shows a residual heart sound presence test in some embodiments of the invention. Frequency adapter 220 locates a first peak (405) in filtered signal waveform 240. Frequency adapter 220 then increments the number of checks performed (410). Frequency adapter 220 then locates a next peak (415). Frequency adapter 220 then determines whether the actual time between the peaks is within a range of expected times between heartbeats (420). In this regard, a nearly periodic heartbeat is expected to have a time-varying period T(t), an average period T and a tolerance Tₜ. If the actual time between the first peak Pᵢ and the next peak Pⱼ is within the tolerance Tₜ, or

|(Pⱼ−Pᵢ)−T|<Tₜ

then the actual time between the peaks is within a range of expected times between heartbeats. If the actual time between the peaks is within the expected range of times between heartbeats, the peaks are potential heartbeats and frequency adapter 220 increments the number of potential heartbeats (425) whereupon frequency adapter 220 selects the next peak as the new first peak (405) and repeats the process. On the other hand, if the actual time between the peaks is less than the expected range of times between heartbeats, the peaks are too close to be potential heartbeats and frequency adapter 220 finds a new next peak (415) to compare with the first peak. Finally, if the actual time between the peaks is greater than the expected range of times between heartbeats, the peaks are too far apart to be potential heartbeats and frequency adapter 220 selects the peak immediately following the first peak as the new first peak (405) and repeats the process. When there are no more next peaks left to compare with the first peak, frequency adapter 220 computes the ratio of potential heartbeats found with the number of checks performed (430). Frequency adapter 220 then compares this ratio with a threshold. If the ratio exceeds the threshold, this is an indication that too much residual heart sound is present in filtered signal waveform 240 and that the cutoff frequency should be reduced.

On the other hand, if the ratio is below the threshold, this is an indication that sufficient heart sound has been removed from filtered signal waveform 240 and that the cutoff frequency does not need to be reduced.

Peak Slopes Test. Lowpass filters having lower cutoff frequencies flatten peaks in an energy envelope more than lowpass filters having higher cutoff frequencies. The peak slopes test compares slope values of peaks in filtered signal waveform 240 with slope values of peaks in input signal waveform 230. If the difference between these slope values exceeds a threshold, this is an indication that too much respiration sound may have been removed from filtered signal waveform 240 and that the cutoff frequency should be increased. On the other hand, if the difference between these slope values is below the threshold, this is an indication that respiration sounds have been adequately preserved and that the cutoff frequency does not need to be increased.

Smoothness Test. Lowpass filters having lower cutoff frequencies remove more high frequency peaks in an energy envelope and make the energy envelope smoother than lowpass filters having higher cutoff frequencies. The smoothness test compares the smoothness of filtered signal waveform 240 with the smoothness of input signal waveform 230 in terms of difference in the number of peaks and/or signal variance. If the difference exceeds a threshold, this is an indication that too much respiration sound may have been removed from filtered signal waveform 240 and that the cutoff frequency should be increased. On the other hand, if the difference is below the threshold, this is an indication that respiration sounds have been adequately preserved and that the cutoff frequency does not need to be increased.

Frequency adapter 220 uses the results of cutoff frequency optimization tests to determine whether the cutoff frequency is too high, too low, or optimal. Where multiple tests are used, frequency adapter 220 combines the results of tests in a “fuzzy” logic process to determine whether the cutoff frequency is too high, too low, or optimal. If the determination is that the cutoff frequency is too high or too low, frequency adapter 220 computes a new cutoff frequency by applying a correction function.

The correction function used to compute the new cutoff frequency may be, by way of example, one of the following:

Frequency Independent Correction. In frequency independent correction, the adjustment made to the cutoff frequency fᵢ₊₁ for the next iteration is independent of the cutoff frequency fᵢ for the prior iteration and the cutoff frequency is computed as

fᵢ₊₁=fᵢ−a(i)

where a cutoff frequency decrease is indicated or

fᵢ₊₁=fᵢ+b(i)

where a cutoff frequency increase is indicated, and where a(i) and b(i) are frequency decrease and increase factors, respectively, which are preferably monotonously decreasing functions. By making adjustments to the cutoff frequency using monotonically decreasing functions rather than constants, eventual convergence at an optimal cutoff frequency is ensured and overshoot is avoided that could lead to unnecessary oscillation and even divergence from an optimal cutoff frequency.

Frequency Dependent Correction. In frequency dependent correction, the adjustment made to the cutoff frequency fᵢ₊₁ for the next iteration is dependent on the cutoff frequency fᵢ for the prior iteration, and the cutoff frequency is computed as

fᵢ₊₁=(1−a(i))fᵢ

where a cutoff frequency decrease is indicated or

fᵢ₊₁=(1+b(i))fᵢ

where a cutoff frequency increase is indicated, and where a(i) and b(i) are frequency decrease and increase factors, respectively, which are preferably monotonously decreasing functions.

Bisectional Correction. In bisectional correction, bounding cutoff frequencies f_{L} and f_{H} are identified at a low limit and a high limit, respectively, and the adjustment made to the cutoff frequency fᵢ₊₁ for the next iteration is

fᵢ₊₁=0.5(fᵢ+f_{L})

where a cutoff frequency decrease is indicated or

fᵢ₊₁=0.5(fᵢ+f_{H})

where a cutoff frequency increase is indicated. If a cutoff frequency decrease is indicated, then f_{H} is replaced in the first equation with fᵢ for the next iteration. Similarly, if a cutoff frequency increase is indicated, then f_{L} is replaced in the second equation with fᵢ for the next iteration.

Once frequency adapter 220 has computed the new cutoff frequency by applying a correction function, frequency adapter 220 sends an instruction 250 to filter stage 210 causing filter stage 210 to set the cutoff frequency to the new cutoff frequency for the next filtering operation performed on input signal waveform 230.

It will be appreciated by those of ordinary skill in the art that the invention can be embodied in other specific forms without departing from the spirit or essential character hereof. The present description is considered in all respects to be illustrative and not restrictive. The scope of the invention is indicated by the appended claims, and all changes that come with in the meaning and range of equivalents thereof are intended to be embraced therein.

What is claimed is:

1. An adaptive acoustic signal filter for a respiration monitoring system, comprising:
a filter stage configured to apply a cutoff frequency to an input acoustic signal waveform containing respiration and heart sounds in a filtering operation to produce a filtered acoustic signal waveform from which heart sounds have been removed; and
a cutoff frequency adapter operatively coupled with the filter stage and configured to perform one or more cutoff frequency optimization tests on the filtered signal, determine from the tests whether adjustment of the cutoff frequency is indicated and selectively adjust the cutoff frequency and provide the adjusted cutoff frequency to the filter stage for application in a next filtering operation performed on the input signal waveform depending on whether adjustment of the cutoff frequency is indicated.

2. The filter of claim 1, wherein the cutoff frequency optimization tests assess whether the filtering operation struck a proper balance between removing heart sounds and preserving respiration sounds in the filtered signal waveform.

3. The filter of claim 1, wherein the cutoff frequency optimization tests comprise a test of residual heart sound presence in the filtered signal waveform.

4. The filter of claim 3, wherein the residual heart sound presence test determines whether actual times between peaks in the filtered signal waveform fall within a range of expected times between heartbeats.

5. The filter of claim 1, wherein the cutoff frequency optimization tests comprise a test of slopes of peaks in the filtered signal waveform.

6. The filter of claim 1, wherein the cutoff frequency optimization tests comprise a test of smoothness of the filtered signal waveform.

7. The filter of claim 1, wherein the cutoff frequency is adjusted using a monotonously decreasing function.

8. The filter of claim 1, wherein the filter is a lowpass filter.

9. The filter of claim 1, wherein the input signal waveform comprises an energy envelope computed from a raw acoustic signal.

10. The filter of claim 1, wherein the filter stage and the cutoff frequency adapter comprise software instructions executed by a processor.

11. An adaptive acoustic signal filtering method for a respiration monitoring system, comprising the steps of:
applying by the system to an input acoustic signal waveform containing respiration and heart sounds in a filtering operation a cutoff frequency to produce a filtered acoustic signal waveform from which heart sounds have been removed;
performing by the system one or more cutoff frequency optimization tests on the filtered signal waveform;
determining by the system from the tests whether adjustment of the cutoff frequency is indicated; and
selectively adjusting by the system the cutoff frequency and providing by the system the adjusted cutoff frequency to the filter for application in a next filtering operation performed on the input signal waveform depending on whether adjustment of the cutoff frequency is indicated.

12. The method of claim 11, wherein the cutoff frequency optimization tests assess whether the filtering operation struck a proper balance between removing heart sounds and preserving respiration sounds in the filtered signal waveform.

13. The method of claim 11, wherein the cutoff frequency optimization tests comprise a test of residual heart sound presence in the filtered signal waveform.

14. The method of claim 13, wherein the residual heart sound presence test determines whether actual times between peaks in the filtered signal waveform fall within a range of expected times between heartbeats.

15. The method of claim 11, wherein the cutoff frequency optimization tests comprise a test of slopes of peaks in the filtered signal waveform.

16. The method of claim 11, wherein the cutoff frequency optimization tests comprise a test of smoothness of the filtered signal waveform.

17. The method of claim 11, wherein the cutoff frequency is adjusted using a monotonously decreasing function.

18. The method of claim 11, wherein the filter is a lowpass filter.

19. The method of claim 11, wherein the input signal waveform comprises an energy envelope computed from a raw acoustic signal.

20. The method of claim 11, wherein the method is performed at least in part by executing software instructions under processor control.
